(19) United States
(12) Patent Application Publication
(10) Pub. No.: US 2020/0230409 A1
(43) Pub. Date: Jul. 23, 2020

(54) LOW-FREQUENCY TREATMENT DEVICE

(71) Applicant: OMRON HEALTHCARE Co., Ltd., Kyoto (JP)

(72) Inventors: Mitsuru SAMEJIMA, Kyoto (JP); Yui WATANABE, Kyoto (JP)

(21) Appl. No.: 16/752,847

(22) Filed: Jan. 27, 2020

Related U.S. Application Data
(63) Continuation of application No. PCT/JP2018/026456, filed on Jul. 13, 2018.

(30) Foreign Application Priority Data
Aug. 10, 2017 (JP) 2017-155103

(51) Int. Cl. A61N 1/36

(52) U.S. Cl. CPC A61N 1/36031; A61N 1/0492

(57) ABSTRACT

A low-frequency treatment device with minimal hindrance to a user's action. The low-frequency treatment device comprises a pad that executes low frequency treatment by applying a low frequency current to a body of a user; and an execution unit that detects vibration of a location where the low frequency current is being applied (step S2) and that, when the vibration is detected, executes prevention processing for preventing the vibration of the location where the low frequency current is being applied; wherein the prevention processing comprises processing for notifying about changing the location where the low frequency current is being applied, for example (step S10).

### CROSS-REFERENCE TO RELATED APPLICATIONS

This is a continuation of International Application PCT/JP2018/026456, with an international filing date of Jul. 13, 2018 and JP 2017-155103 filed on Aug. 10, 2017, filed by applicant, the disclosure of which is hereby incorporated by reference in its entirety.

### TECHNICAL FIELD

The present disclosure relates to a low-frequency treatment device.

### BACKGROUND ART

Known low-frequency treatment devices are configured to apply electrical stimulation to an area of the user's body by outputting a low-frequency pulse to the area via an electrode pad (see Patent Document 1).

### CITATION LIST

### Patent Literature

Patent Document 1: JP 2005-152412 A

### SUMMARY OF INVENTION

### Technical Problem

With the low-frequency treatment device of Patent Document 1, the site where electrical stimulation is being applied may vibrate (wobble). When the user performs actions, the vibration may be a hindrance to the action. For example, when the user is walking, the vibration may encumber walking.

The present disclosure has been made in view of the circumstances described above, and a low-frequency treatment device with minimal hindrance to a user's action according to an embodiment is described.

### Solution to Problem

A low-frequency treatment device according to this disclosure includes

a treatment portion that executes low frequency treatment by applying a low frequency current to a body of a user;

a detection unit that detects vibration of a location where the low frequency current is being applied; and

an execution unit that, when the vibration is detected, executes prevention processing for preventing the vibration of the location where the low frequency current is being applied.

In some embodiments, the prevention processing includes processing for notifying about changing the location where the low frequency current is being applied.

In some embodiments, the low-frequency treatment device includes a plurality of the treatment portions, wherein

the prevention processing includes processing to switch a treatment portion to apply the low frequency current from a treatment portion of the plurality of treatment portions that is applying the low frequency current to another treatment portion of the plurality of treatment portions, when the vibration is detected.

In some embodiments, the prevention processing includes processing to control the low frequency current applied and reduce a strength of the low frequency current.

In some embodiments, the detection unit detects the vibration by detecting whether an acceleration of the location where the low frequency current is being applied is greater than a threshold.

In some embodiments, a first reception unit is further provided that receives a setting of the threshold.

In some embodiments, a second reception unit is further provided that receives from a user a mode that is set from among a plurality of modes including

a first mode in which the execution unit executes the prevention processing when the vibration is detected, and

a second mode in which the execution unit does not execute the prevention processing when the vibration is detected.

### Advantageous Effects of Invention

According to this disclosure, minimal hindrance is caused to a user's action.

### BRIEF DESCRIPTION OF DRAWINGS

FIG. 1 is a diagram illustrating the appearance of a low-frequency treatment device 20 in association with a terminal device 10.

FIG. 2 is a diagram schematically illustrating the internal configuration of the low-frequency treatment device 20.

FIG. 3 is a configuration diagram of the terminal device 10.

FIG. 4 is a diagram illustrating a flowchart involving a low-frequency treatment device and a terminal device.

FIG. 5 is a diagram illustrating an example of a display of a display 1.

FIGS. 6A and 6B are diagrams illustrating a low-frequency treatment device according to another embodiment.

FIG. 7 is a diagram schematically illustrating the internal configuration of a low-frequency treatment device according to another embodiment.

FIG. 8 is a diagram illustrating a flowchart of a low-frequency treatment device according to another embodiment.

FIG. 9 is a diagram illustrating a flowchart of a low-frequency treatment device according to another embodiment.

FIG. 10 is a diagram illustrating reception of a mode selection.

FIG. 11 is a diagram illustrating reception of a threshold input.

### DESCRIPTION OF EMBODIMENTS

Embodiments will be described below with reference to the drawings. The same reference numerals are assigned to the same components and corresponding components, and redundant descriptions may not be repeated.

### First Embodiment

### System Configuration

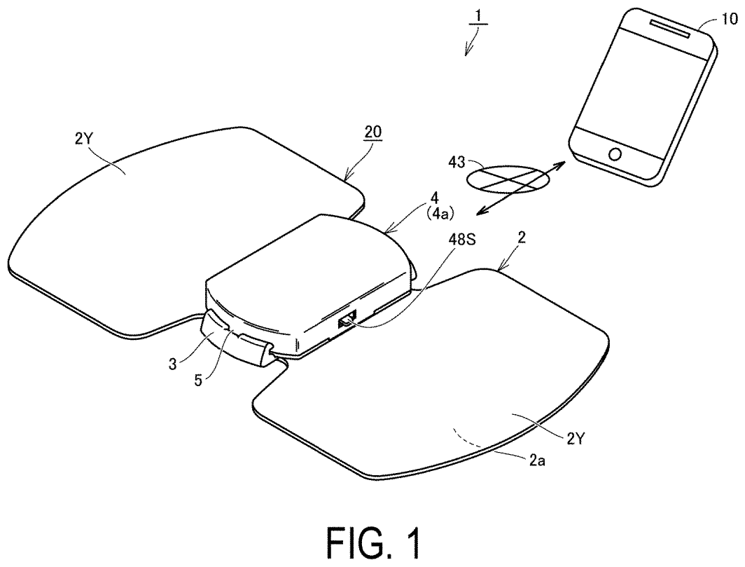

FIG. 1 is a diagram illustrating a schematic configuration of a treatment system 1 according to a first embodiment. Referring to FIG. 1, the treatment system 1 includes a terminal device 10 used by a user, a low-frequency treatment device 20, and a network 43. The terminal device 10 is an example of an information processing device. The terminal device 10 is, for example, a smart phone including a touch panel. Note that the terminal device 10 may be another type of terminal device such as a folding type mobile telephone, a tablet terminal device, a personal computer (PC) and a personal data assistant (PDA).

The network 43 employs a short-range wireless communication system, typically Bluetooth (trademark) low energy (BLE), to connect the terminal device 10, the low-frequency treatment device 20. The network 43 is not limited thereto, and a wired communication system may be employed, or other wireless communication systems such as a wireless local area network (LAN) may be employed.

The low-frequency treatment device 20 includes a pad 2, a holder 3, and a body portion 4. The pad 2 is also referred to as a treatment portion. The low-frequency treatment device 20 is a so-called cordless type and is controlled according to control information received from the terminal device 10.

### Pad 2

The pad 2 is an example of an electrode portion that is configured to come into contact with an area of the body. The pad 2 has a sheet-like shape and is attached to an area of the user's body, specifically to the area to be treated or the like. A conductive layer 2a is provided on a surface (lower surface) of the outer surface of the pad 2 facing the body. The pad 2 is attached to the user's skin using a conductive gel or the like, and a pulse current at a frequency corresponding to the treatment program is supplied to the user through the conductive layer 2a.

The pad 2 includes an attachment portion (not illustrated) and a treatment portion 2Y. The attachment portion is held by the holder 3. The holder 3 is positioned and disposed in the attachment portion. The treatment portion 2Y is provided on both the left and right sides of the attachment portion, and the conductive layer 2a is exposed on the surface of the treatment portion 2Y that faces the body. The conductive layer 2a is also exposed on the surface facing the body portion 4, and the exposed portion constitutes an electrode.

### Body Portion 4

As illustrated in FIG. 1, the body portion 4 includes a case 4a with a substantially rectangular parallelepiped shape as an outer cover. An engagement portion 5 is formed between the case 4a and the holder 3, and the body portion 4 (case 4a) is detachably attached to the holder 3 by the engagement portion 5. The body portion 4 is provided with a switch 48S that is operated by a user to control the low-frequency treatment device 20. The body portion 4 also includes a display unit (not illustrated) for outputting information such as an operating state of the low-frequency treatment device 20.

With the main body portion 4 attached to the holder 3, the main body portion 4 supplies a low-frequency pulse current to the conductive layer 2a of the pad 2. Specifically, the body portion 4 includes a built-in substrate, electric circuit mounted on the substrate, and the like.

### Circuit Configuration of Low-frequency Treatment Device 20

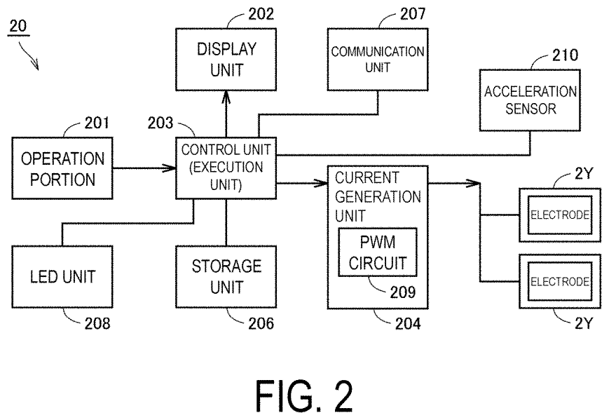

FIG. 2 is a diagram schematically illustrating the internal configuration of the low-frequency treatment device 20 according to the first embodiment. With reference to FIG. 2, the low-frequency treatment device 20 may include, as a primary component, an operation portion 201, a display unit 202, a control unit 203 (also referred to as an execution unit) including a central processing unit (CPU), a current generation unit 204 including a pulse width modulation (PWM) circuit 209, a storage unit 206, a communication unit 207, a light emitting diode (LED) unit 208, and an acceleration sensor 210. Although not illustrated, the low-frequency treatment device 20 includes a power source unit for supplying power to each component. An alkaline battery, for example, may be used as the power source unit. The power source unit generates a drive voltage that stabilizes the battery voltage and supplies it to each component.

The control unit 203 typically includes a central processing unit (CPU) or a microprocessor unit (MPU). The control unit 203 functions as a control unit that controls the operation of the components of the low-frequency treatment device 20 by reading out and executing the program stored in the storage unit 206. By executing the treatment program, the control unit 203 generates a PWM control signal (such as a voltage signal) according to the modified parameter and outputs the generated signal to the PWM circuit 209. The control unit 203 also functions as an execution unit that executes the prevention processing described below.

The storage unit 206 is realized by random access memory (RAM), read-only memory (ROM), flash memory, and the like. The storage unit 206 stores programs (including a treatment program) executed by the control unit 203, data used by the control unit 203, and the like.

The operation portion 201 includes, for example, the switch 48S and various switches to accept a user's operation input to the low-frequency treatment device 20. When the user operates the operation portion 201, the control unit 203 receives the operation content and controls each unit according to the received operation content.

The communication unit 207 includes a circuit for communicating with the terminal device 10. The communication unit 207 receives control information from the terminal device 10 and outputs the control information to the control unit 203. The control information includes the above-described parameters and a command instructing to set the treatment program with the parameters.

The current generation unit 204 is a circuit that generates a current (hereinafter, also referred to as a treatment current) that flows from the pad 2 to the area of the user's body (specifically, the area to be treated). The PWM circuit 209 of the current generation unit 204 outputs a pulse current having an amplitude, a duty ratio (cycle), and the like indicated by the PWM control signal (i.e., the parameters), while keeping the pulse current constant and adjusting the output current amount in accordance with the PWM control signal from the control unit 203. In this way, the waveform of the treatment current output from the current generation unit 204 (hereinafter, also referred to as a treatment waveform) is determined by the PWM control signal according to a parameter from the control unit 203. Note that the treatment current is not limited to a pulse current, and an alternating current may be used, or both types of current may be selectively used.

The treatment current output by the current generation unit 204 includes, for example, a pulse current with a low frequency. The pulse width of the treatment waveform with a low frequency pulse current may be, for example, about 100 μsec for a small pulse width. Also, the maximum pulse amplitude is approximately 100 V, for example. The current generation unit 204 can alter the pulse waveform (amplitude, pulse width, period, and the like) according to the treatment program to provide various types of stimulation to the area, such as “massage”, “tap”, “press” and “rub” and the like.

In addition, by changing the parameters of the treatment program, the treatment content for the area can be changed (adjusted). In particular, changing the amplitude or pulse width of a pulse allows the adjustment of the “intensity” (electrical stimulation intensity) of the stimulation, such as “massage”, “tap”, “press” and “rub” and adjustment of the “speed” of the stimulation, such as “massage”, “tap”, “press” and “rub”.

The acceleration sensor 210 detects the acceleration of the low-frequency treatment device 20. The detected acceleration is input to the control unit 203.

### Configuration of Terminal Device 10

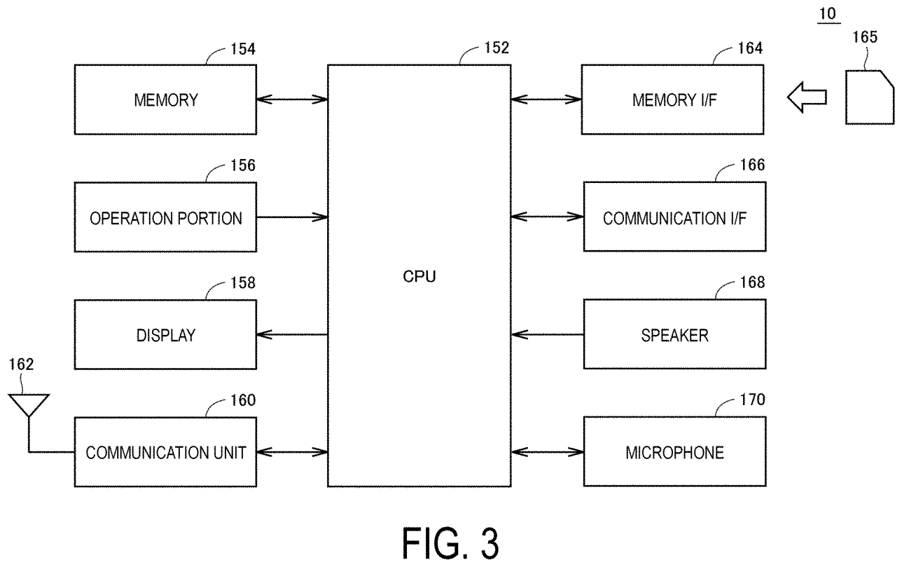

FIG. 3 is a configuration diagram of the terminal device 10 according to the first embodiment. Referring to FIG. 3, the terminal device 10 includes, as main components, a CPU 152, a memory 154, an operation portion 156 that receives a user's operation to the terminal device 10, a display 158, a communication unit 160 for wireless communication via an antenna 162, a memory interface (I/F) 164, a communication interface (I/F) 166, a speaker 168 for audio output, and a microphone 170 for voice input.

The CPU 152 controls each unit by executing programs stored in the memory 154.

A storage region of the memory 154 may be constituted by RAM, ROM, flash memory, a hard disk device, and/or the like. Programs executed by the CPU 152, data used by the CPU 152, and the like are stored in the memory 154.

The operation portion 156 receives an operation input to the terminal device 10. Typically, the operation portion 156 includes a touch panel. The touch panel is provided on the display 158. The operation portion 156 may include a switch button or the like.

The communication unit 160 connects to a mobile communication network via the antenna 162 and transmits and receives signals for wireless communication. Accordingly, the terminal device 10 can communicate with other communication devices (for example, the server 30 and the other terminal device 10) via a mobile communication network such as long term evolution (LTE), for example.

The memory interface 164 reads data from an external storage medium 165. The CPU 152 reads the data stored in the storage medium 165 via the memory interface 164 and stores the data in the memory 154. The CPU 152 reads the data from the memory 154 and stores the data in the external storage medium 165 via the memory interface 164.

The storage medium 165 may include a medium that stores programs or data in a non-volatile manner, such as a compact disc (CD), digital versatile disk (DVD), universal serial bus (USB) memory, and secure digital (SD) memory card.

The communication interface (I/F) 166 is realized by an adapter, connector, or the like to control communication between the terminal device 10 and the low-frequency treatment device 20. In the present embodiment, BLE is employed as the communication method. However, the communication method may be a wireless communication method such as wireless LAN or a wired communication method using universal serial bus (USB) or the like.

### Application Location of Low-Frequency Treatment Device 20

Hereinafter, the location on the user's body where the pad 2 of the low-frequency treatment device 20 is attached and where a low frequency current is applied to the user's body is referred to as an “application location”. The application location includes a location where a low frequency current is output from one of the set of pads 2, and a location where the output low frequency current returns to the other one of the set of pads 2 after passing through the user's body. That is, the application location may also be referred to as a location through which a low frequency current runs. The application location may also be referred to as a location where one of the set of pads 2 is attached.

Having the pulse width of the treatment current at a relative low value (for example, 60 μs) and attaching the pads 2 of the low-frequency treatment device 20 at intended locations intended by the maker of the low-frequency treatment device 20 allows a low frequency treatment that does not contract the user's muscles and has a high therapeutic effect to be executed. However, if the application location is different from the intended location, the treatment current output by the low-frequency treatment device 20 may cause the application location to vibrate. Hereinafter, this vibration is also referred to as “wobble”. For example, in the case where the application location is on the skin above a muscle, the application location tends to vibrate.

When the application location vibrates, the action of the user is hindered. For example, in the case where the application location is the foot or leg of the user and the application location vibrates, the user walks with the application location vibrating, hindering the walking action of the user.

Thus, the low-frequency treatment device 20 executes a prevention processing for preventing vibration at the application location. The prevention process of the present first embodiment is processing for notifying about changing the application location of the pad 2. Furthermore, the acceleration sensor 210 of the present embodiment detects the acceleration due to vibration of the application location of the user and the acceleration of application location due to the user's action (for example, the user walking).

Furthermore, when the application location vibrates, the pad 2 is in contact with the application location, so the vibration is also transmitted to the main body portion 4. Accordingly, the acceleration sensor 210 internally provided in the main body portion 4 can detect the acceleration due to the transmitted vibration (acceleration of the low-frequency treatment device 20) as the acceleration due to the vibration of the application location.

### Processing of Treatment System 1

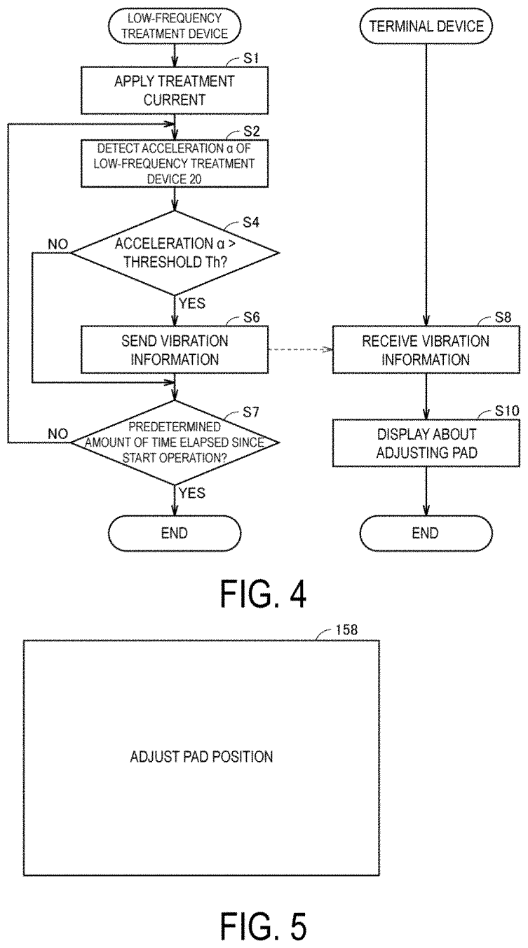

FIG. 4 illustrates the processing flow of the treatment system 1. When the user performs the start operation of low frequency treatment by the low-frequency treatment device 20, the processing of FIG. 4 starts.

First, in step S1, a treatment current from the electrode is applied. Next, in step S2, the acceleration sensor 210 detects an acceleration a of the low-frequency treatment device 20 as the acceleration a of the application location. Then, in step S4, the control unit 203 determines whether the detected acceleration a is greater than a preset threshold Th. The acceleration a being greater than the threshold Th means that the application location is vibrating (wobbling). The acceleration a being equal to or less than the threshold Th means that the application location is not vibrating.

If NO is determined in step S4, i.e., the application location is determined to be not vibrating, the processing of the low-frequency treatment device 20 moves to step S7. In step S7, the control unit 203 determines whether a predetermined amount of time has elapsed since the user executed the start operation of the low frequency treatment. “Predetermined amount of time” refers to the amount of time from the execution of the start operation during which the application location is not wobbling, for example, 10 seconds. In step S7, processing of the low-frequency treatment device 20 is stopped if the predetermined amount of time has been determined to have elapsed. Also, in step S7, processing of the low-frequency treatment device 20 returns to step S2 if the predetermined amount of time has been determined to have not elapsed. If YES is determined in step S4, i.e., the application location is determined to be vibrating, the processing of the low-frequency treatment device 20 moves to step S6.

In step S6, the control unit 203 generates vibration information indicating that the acceleration a is greater than the threshold Th, i.e., the application location is vibrating. In step S6, the control unit 203 sends the vibration information to the terminal device 10.

In step S8, the terminal device 10 receives the vibration information. In step S10, the terminal device 10 causes the display 158 to display change information that indicates that the location where the pad 2 is attached should be changed.

FIG. 5 illustrates an example of the change information displayed on the display 158. In the example of FIG. 5, the change information includes the characters “Adjust the position of the pad”. In this way, the low-frequency treatment device 20 executes processing to cause the display 158 of the terminal device 10 to display the characters “Adjust the position of the pad” as the processing for notifying about changing the location where the pad is attached.

Accordingly, the low-frequency treatment device 20 of the first embodiment causes the display 158 of the terminal device 10 to display the characters “Adjust the position of the pad” as the prevention processing when vibration of the application location of the pad 2 is detected. Thus, the control unit 203 can prompt the user to adjust the position of the pad. The user then can adjust the position of the pad to prevent vibration (wobble) of the application location on the user's body.

The acceleration sensor 210 and the pad 2 attachable to the user's body are provided in the same device (the low-frequency treatment device 20). Accordingly, the acceleration sensor 210 can detect the vibration of the application location of the pad 2 as the vibration of the low-frequency treatment device 20. Thus, the acceleration sensor 210 can detect the acceleration of the application location of the pad 2 with precision.

The acceleration sensor 210 of the present embodiment is not required to detect the acceleration due to vibration of the application location of the user distinctively from the acceleration of application location due to the user's action (for example, the user walking). That is, the relatively cheap acceleration sensor 210 can be used. For example, take the case where the user walks while receiving low frequency treatment and the acceleration sensor 210 detects acceleration due to walking despite the application location not vibrating. In this case, even though the application location is not vibrating, a false positive notification about change information may be issued. However, in the present embodiment, as illustrated in step S7 of FIG. 4, the processing of step S2, step S4, and step S6 are executed while the predetermined amount of time from the start operation has not elapsed and are not executed after the predetermined amount of time has elapsed. “Predetermined amount of time” refers to the amount of time from the execution of the start operation during which the application location is not wobbling. In this way, the low-frequency treatment device 20, by executing the processing of step S7, can prevent such false positives.

Furthermore, the “processing for notifying about changing the application location” may be a different processing. An example of a different processing includes processing to display the change information on a display portion (not illustrated) of the low-frequency treatment device 20. Another example of a different processing includes processing to output a sound indicating the change information from the terminal device 10 and/or the low-frequency treatment device 20.

### Second Embodiment

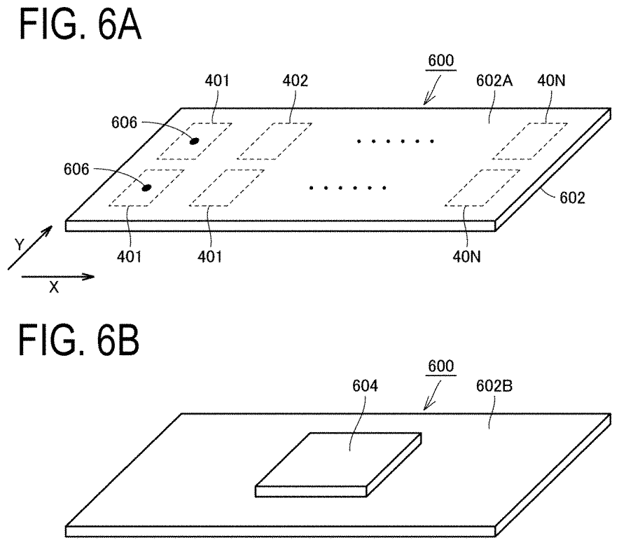

Next, a second embodiment will be described. FIGS. 6A and 6B are diagrams illustrating the appearance of a low-frequency treatment device 600 according to the second embodiment. FIG. 6A illustrates the side of the low-frequency treatment device 600 with electrodes. FIG. 6B illustrates the side of the low-frequency treatment device 600 opposite the side illustrated in FIG. 6A.

Referring to FIG. 6A, the low-frequency treatment device 600 includes a rectangular member 602 with a rectangular shape. The extension direction (longitudinal direction) of the rectangular member 602 is the X direction and the width direction is the Y direction. N number (N being an integer two or greater) of pairs of electrodes (positive electrode and negative electrode) are disposed in the rectangular member 602. Hereinafter, a pair of electrodes is also referred to as an “electrode pair”.

In the example of FIG. 6A, the two electrodes of the electrode pairs are arranged in the Y direction. The electrode pairs are arranged in the X direction. Note that the arrangement of the two electrodes of the electrode pairs and the arrangement of the electrode pairs are not limited to this arrangement, and in modified examples a different arrangement may be employed.

The low-frequency treatment device 600 includes N number of electrode pairs 401, 402, . . . , 40N. Also, the surface illustrated in FIG. 6A is an attachment surface 602A that is attachable to the user's body. Any technique may be used for attaching the attachment surface 602A to the user's body. For example, a portion or all of the attachment surface 602A may be adhesive. Any technique may be used for conferring adhesiveness. For example, a portion or all of the attachment surface 602A may be the portion with adhesiveness. Alternatively, an adhesive member with adhesiveness may be provided on a portion of all of the attachment surface 602A. The attachment surface 602A of the low-frequency treatment device 600 can be attached to the user's body via adhesion.

Furthermore, a technique for attaching the attachment surface 602A to the user's body may include employing an attachment member. The attachment member may be, for example, a suction cup attached to the user's body. The low-frequency treatment device 600 may include a pair of mounting members on both ends of the rectangular member. The pair of mounting members may be, for example, magic tape (trade name) or another hook-and-loop fastener. The user can attach the low-frequency treatment device 600 by, for example, wrapping it around their wrist or ankle and fastening together the pair of mounting members on the ends.

The N number of electrodes also include one initial electrode pair. In the example of FIG. 6A, an electrode pair 401 is the initial electrode pair. Also, marks 606 are located at positions where the electrode pair 401 are internally provided. The mark 606 may be any mark, and in the example of FIG. 6A, it is a black dot. When treatment by the low-frequency treatment device 600 starts, a treatment current is output through the initial electrode pair. The user may align the marks 606 with the desired low frequency treatment position and attach the low-frequency treatment device 600 to the body.

Also, as illustrated in FIG. 6B, a main body portion 604 is disposed on a placement surface 602B on the side opposite the attachment surface 602A of the low-frequency treatment device 600.

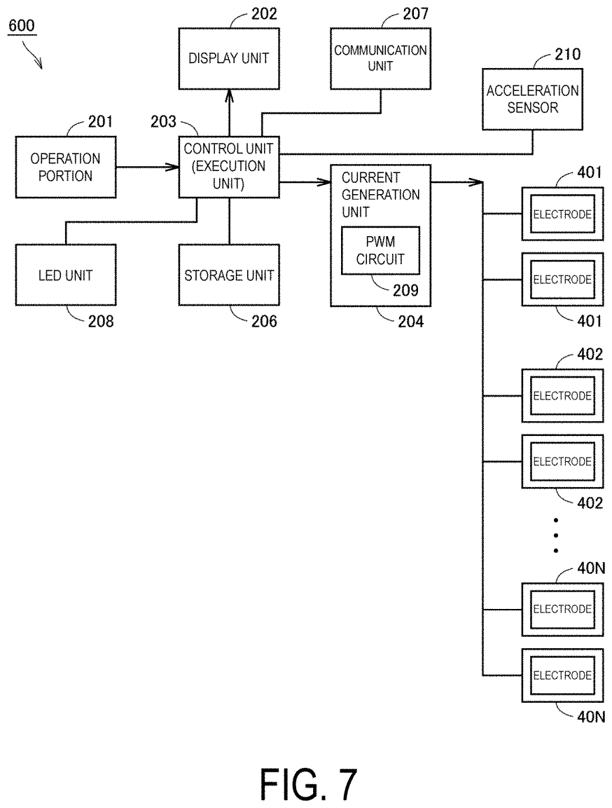

FIG. 7 is a diagram schematically illustrating the internal configuration of the low-frequency treatment device 600 according to the second embodiment. The difference between the drawings FIG. 7 and FIG. 2 is that in FIG. 2 there is one electrode pair while in FIG. 7 there are N number of electrode pairs. Furthermore, by controlling the current generation unit 204, the control unit 203 can sequentially activate the electrode pairs used in outputting the treatment current. In other words, by controlling the current generation unit 204, the control unit 203 can sequentially activate the positive electrodes that output the treatment current of the N number of electrode pairs 401, 402, . . . , 40N.

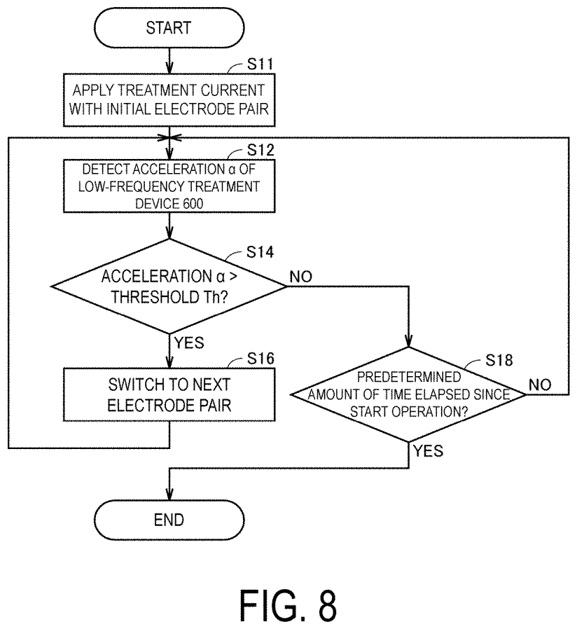

FIG. 8 is a diagram illustrating the processing flow of the low-frequency treatment device 600 according to the second embodiment. When the user performs the start operation of low frequency treatment by the low-frequency treatment device 600, the processing of FIG. 8 starts.

First, in step S11, the low-frequency treatment device 600 outputs a treatment current using the initial electrode pair. Next, in step S12, the acceleration sensor 210 detects an acceleration a of the low-frequency treatment device 600 as the acceleration a of the application location due to the initial electrode pair.

In step S14, the control unit 203 determines whether the detected acceleration a is greater than a preset threshold Th. The acceleration a being greater than the threshold Th means that the application location is vibrating (wobbling) due to the initial electrode pair. The acceleration a being equal to or less than the threshold Th means that the application location is not vibrating due to the initial electrode pair.

If NO is determined in step S14, i.e., the application location is determined to be not vibrating due to the initial electrode pair, the processing of the control unit 203 moves to step S18. In step S18, the control unit 203 determines whether a predetermined amount of time has elapsed since the user executed the start operation of the low frequency treatment. “Predetermined amount of time” refers to the amount of time from the execution of the start operation during which the application location is not wobbling, for example, 10 seconds. In step S18, processing is stopped if the control unit 203 determines that the predetermined amount of time has elapsed. Also, in step S18, processing of the control unit 203 returns to step S12 if the control unit 203 determines that predetermined amount of time has not elapsed. If YES is determined in step S14, i.e., the application location is determined to be vibrating due to the initial electrode pair, the processing of the control unit 203 moves to step S16.

In step S16, the control unit 203 switches the electrode pair used to output a treatment current to the next electrode pair. The post-switch electrode pair is used to output a treatment current. That is, in step S16, if YES is determined for step S14, the control unit 203 stops the output of a treatment current from the electrode pair outputting the treatment current and causes the next electrode pair to start outputting a treatment current. Also, “next electrode pair” refers to the electrode pair with a reference number one greater than that of the electrode pair that had been outputting the treatment current. For example, if YES is determined for step S14, the electrode pair outputting the treatment current switches from, for example, electrode pair 402 that had been outputting the treatment current to the next electrode pair 403. When the processing of step S16 ends, the processing of the control unit 203 returns to step S12.

In step S12, the control unit 203 detects an acceleration a of the low-frequency treatment device 600 as the vibration of the application location due to the electrode pair switched to in step S16. When the processing of step S12 ends, the control unit 203 executes the processing of step S14 and step S16. The processing loop of step S12, step S14, and step S16 ends when NO is determined for step S14. NO being determined for step S14 means that the acceleration of the application location due to the electrode pair switched to in step S16 is equal to or less than the threshold.

In this way, when vibration of the application location due to the electrode pair is detected, the low-frequency treatment device 600 of the second embodiment, as prevention processing, switches the electrode pair used to output the low frequency current. Accordingly, vibration of the application location can be prevented without the user moving the low-frequency treatment device 600. Thus, the user convenience can be enhanced.

Also, in step S16, the low-frequency treatment device 600 switches from the pre-switch electrode pair to the electrode pair located nearby. Accordingly, the low-frequency treatment device 600 can apply a treatment current to a location as close as possible to the user's desired location. Thus, even if the electrode pair applying the treatment current is switched, the low-frequency treatment device 600 can keep user dissatisfaction to a minimum.

### Third Embodiment

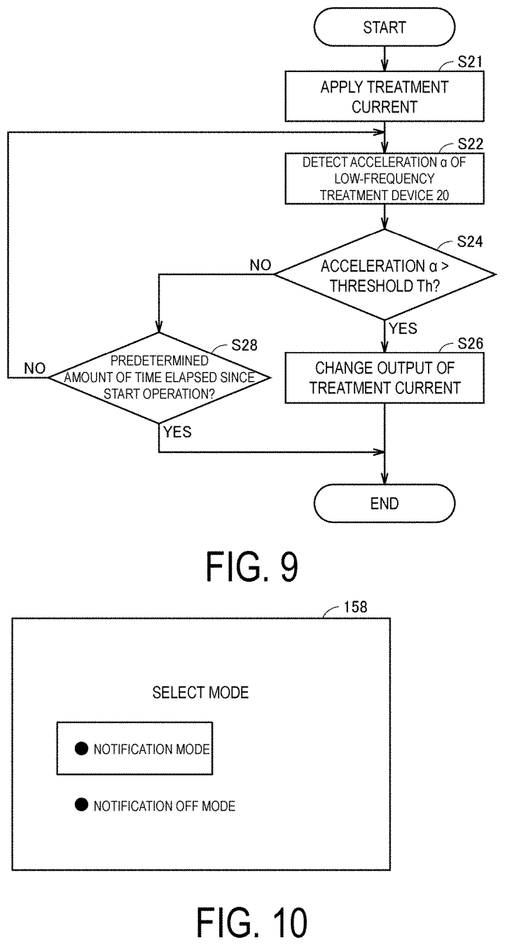

Next, a third embodiment will be described. The appearance of the low-frequency treatment device and internal configuration of the low-frequency treatment device of a treatment system of the third embodiment are similar to that illustrated in FIGS. 1 and 2. FIG. 9 is a diagram illustrating the processing flow of the low-frequency treatment device 20 according to the third embodiment.

The processing of step S21, step S22, and step S24 is the same as the processing of step S1, step S2, and step S4 of FIG. 4. If YES is determined in the processing of step S24, the processing moves to step S26. In step S26, the output of the treatment current is changed so as to eliminate the vibration of the application location. In step S26, for example, processing is executed to control the application of the low frequency current to reduce the intensity of the low frequency treatment. For example, the waveform of the low frequency current includes three parameters. The three parameters include amplitude, pulse width, and pulse frequency. By changing at least one parameter of the three parameters, the intensity of the low frequency treatment can be reduced. For example, reducing the amplitude reduces the intensity of the low frequency treatment.

In this way, when vibration of the application location due to the pad 2 is detected, the low-frequency treatment device 20 of the third embodiment, as prevention processing, executes processing to control the application of the low frequency current to reduce the intensity of the low frequency treatment. This can prevent vibration (wobble) of the application location on the user's body.

### OTHER EMBODIMENTS

(1) The embodiments described above may include a first mode and a second mode selectable by the user. In the description below, the prevention processing is any of the prevention processing described in the first to third embodiments. The first mode is a mode in which the prevention processing is executed when vibration is detected. The second mode is a mode in which the prevention processing is not executed when vibration is detected. For example, mode selection from the operation portion 156 of the terminal device 10 can be received by a second reception unit.

FIG. 10 illustrates an example of a mode selection screen in the case where the prevention processing is processing to notify with change information. In the example of FIG. 10, a notification mode is displayed as the first mode, and a notification off mode is displayed as the second mode. The notification mode is a mode in which change information is notified of when the application location vibrates. The notification off mode is a mode in which change information is not notified of when the application location vibrates.

By moving a cursor 158A, the user can set the mode to notification mode or notification off mode.

For example, a user desiring to be notified of change information when the application location vibrates can set the mode to the first mode. A user desiring not to be notified of change information when the application location vibrates can set the mode to the second mode if they feel annoyed by the change information notification.

In this way, the user can select the mode, thus further enhancing user convenience.

(2) In the embodiments described above, the threshold Th is a preset value. However, the threshold Th may be able to be set by the user. For example, mode selection from the operation portion 156 of the terminal device 10 can be received by a first reception unit.

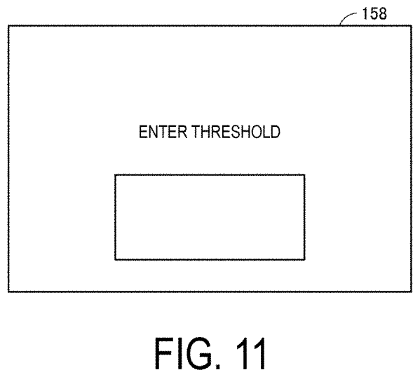

For example, a user who is not worried by slight wobbling may set the threshold Th to a high value. A user who is worried by slight wobbling may set the threshold Th to a low value. FIG. 11 is a diagram illustrating an example of an input screen where the threshold Th can be input. The input screen is displayed on the display 158. This setting can be received from the operation portion 201 or the operation portion 156, for example. Such a configuration allows the user to set the threshold Th. Thus, the user convenience can be further enhanced.

### Modified Example

(1) In the embodiments described above, vibration (wobble) of the application location is detected by the acceleration sensor 210. However, vibration of the application location may be detected by another device. For example, vibration of the application location may be detected using a myoelectric sensor that detects action potential produced when muscles contract. A configuration may be employed in which, in the case where the action potential of the application location is determined to be equal to or greater than the predetermined threshold, the application location is determined to be vibrating. In another possible configuration, an action potential pattern for when muscles or the like are vibrating is obtained in advance as a vibration pattern, and, when the action potential pattern of the application location is similar to or matches the vibration pattern, the application location is determined to be vibrating. Also, the myoelectric sensor may be provided integrally with the low-frequency treatment device (in the same case) or may be provided separate from the low-frequency treatment device (in a different case).

Furthermore, the control unit may detect vibration of the application location using both the myoelectric sensor and the acceleration sensor. For example, when vibration of the application location is detected by both the myoelectric sensor and the acceleration sensor, the control unit 203 determines that the application location is vibrating. The control unit 203 determines that the application location is not vibrating when vibration of the application location is detected by either the myoelectric sensor or the acceleration sensor or when vibration of the application location is detected by neither the myoelectric sensor nor the acceleration sensor. Furthermore, another device may be used together with the myoelectric sensor and the acceleration sensor to detect vibration of the application location.

In this manner, by two or more devices detecting vibration of the application location, the precision of detecting vibration of the application location can be enhanced.

(2) The acceleration sensor described above detects the acceleration due to vibration of the application location of the user and the acceleration of application location due to the user's action (for example, the user walking). That is, the acceleration sensor described above detects the acceleration due to small movement and the acceleration due to large movement. However, an acceleration sensor of a modified example may detect the acceleration of small movement (vibration of the application location of the user) without detecting the acceleration due to large movement (user action) via filtering processing or the like. Using such an acceleration sensor allows the low-frequency treatment device described above to be used regardless of whether the user is walking or not.

(3) The low-frequency treatment device of the present embodiment described above is a cordless type. However, the low-frequency treatment device may be a wired type. Furthermore, the acceleration sensor described above is internally provided in the main body portion 4. However, the acceleration sensor may be provided external to the main body portion 4. In such a configuration, the detection results of the acceleration sensor are sent to the main body portion 4 via a wired or wireless connection.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

20 Low-frequency treatment device
30 Server
43 Network
203 Control unit
204 Current generation unit
206 Storage unit

1. A low-frequency treatment device, comprising:
a treatment portion configured to execute low frequency treatment by applying a low frequency current to a body of a user;
a detection unit configured to detect vibration of a location where the low frequency current is being applied; and
an execution unit configured to, when the vibration is detected, execute prevention processing for preventing the vibration of the location where the low frequency current is being applied; wherein
the prevention processing comprises processing for notifying about changing the location where the low frequency current is being applied.

2. The low-frequency treatment device according to claim 1, further comprising a plurality of the treatment portions, wherein
the prevention processing comprises processing to switch a treatment portion to apply the low frequency current from a treatment portion of the plurality of treatment portions that is applying the low frequency current to another treatment portion of the plurality of treatment portions, when the vibration is detected.

3. The low-frequency treatment device according to claim 1, wherein
the prevention processing comprises processing to control the low frequency current applied and reduce a strength of the low frequency current.

4. The low-frequency treatment device according to claim 1, wherein the detection unit is configured to detect the vibration by detecting whether an acceleration of the location where the low frequency current is being applied is greater than a threshold.

5. The low-frequency treatment device according to claim 4, further comprising a first reception unit configured to receive a setting of the threshold.

6. The low-frequency treatment device according to claim 1, further comprising a second reception unit configured to receive from a user a mode that is set from among a plurality of modes comprising
a first mode configured to allow the execution unit to execute the prevention processing when the vibration is detected, and
a second mode configured not to allow the execution unit to execute the prevention processing when the vibration is detected.
